(19) United States
(12) Patent Application Publication
(10) Pub. No.: US 2012/0009593 A1
(43) Pub. Date: Jan. 12, 2012

(54) ANALYSIS METHOD FOR THE IN VITRO DIAGNOSIS OF BENIGN PROSTATIC HYPERPLASIA (BPH) IN DOGS BY ASSAYING CANINE PROSTATE SPECIFIC ESTERASE, AND BPH TREATMENT FOLLOW UP

(75) Inventors: Emmanuel Claret, Rochefort du Gard (FR); Stephane Audhuy, La Seyne Sur Mer (FR); Jerome Morlet, Sollies Pont (FR); Gerard Papierok, Hyeres Les Palmiers (FR)

(73) Assignee: VIRBAC SA, Carros (FR)

(21) Appl. No.: 13/202,930

(22) PCT Filed: Feb. 23, 2010
(86) PCT No.: PCT/FR10/00172

§ 371 Date: Sep. 23, 2011

(30) Foreign Application Priority Data
Feb. 23, 2009 (FR) 0900809

(51) Int. Cl. G01N 33/573; C12N 9/96; C07K 16/40; G01N 33/577

(52) U.S. Cl. 435/7.4; 435/188; 530/391.3

(57) ABSTRACT

The invention relates to an analysis method for the in vitro diagnosis of benign prostatic hyperplasia (BPH) in a dog, using the CPSE contained in biological fluid samples collected from the dog as well as biological molecules specifically binding CPSE. The invention is characterised in that it includes the following steps: a. collecting a biological fluid sample from a dog; b. measuring the canine prostate specific esterase (CPSE) of said sample; c. comparing said measured CPSE concentration with a reference value, said value being from around 34 ng/ml to around 102.4 ng/ml for serum or plasma, preferably around 61 ng/ml; d. using the CPSE concentration as a BPH marker; and e. diagnosing BPH in a dog when measured CPSE concentrations in said sample exceed said reference value.

The invention relates to an analysis method for the in vitro diagnosis of benign prostatic hyperplasia (BPH) in dogs, in particular by assaying the Canine Prostate Specific Esterase (CPSE) parameter in various biological fluids such as, for example, urine, seminal fluid, blood. This method can distinguish not only PBHs in normal prostates, but also PBHs in the cases of more severe conditions of the canine prostate such as prostatitis and adenocarcinoma. The invention can also be applied to the follow up of a BPH treatment.

### PRIOR ART

Benign prostatic hyperplasia (BPH) is a very common condition in older adult dogs: from histological analysis, from 50 to 80% of dogs over the age of 5 suffer from hyperplasia and prostatic hypertrophy (Johnston S D, Kustritz M V R, Oison P N S. Benign Prostatic Hypertrophy/Hyperplasia. In: Canine and feline theriogeology. Philadelphia: WB Saunders, 2001, 340-341). In most cases, BPH has no consequences for the dog. However, BPH can lead to clinical manifestations when it becomes severe (a size 2-6 times larger than normal), and facilitate the emergence of other more serious prostatic disorders (prostatitis, large cysts, prostatic abscesses, etc.).

It is therefore essential for the practitioner to be able to diagnose BPH as early as possible in order to prevent the development of lesions and the appearance of marked symptoms.

BPH is a chronic disorder if the dog has not been neutered: the prostate gradually grows in the absence of any treatment or after treatment discontinuation. To avoid the worsening or return of the symptoms, it is important for the veterinarian to be able to monitor the evolution of the prostate.

Current methods for diagnosing BPH include digital prostatic examination, radiography and ultrasonography. However, these three methods do not provide satisfactory results.

The digital prostatic examination is an examination which has many limitations, in particular:
for the operator: who must palpate many healthy prostates before being able to identify BPH;
for the dog: digital rectal examination is impossible in large and miniature breeds. Moreover, there are racial specificities, which need to be understood (Scottish-Terrier, etc.);
for the examination itself: palpation is restricted to the dorsal surface of the prostate, which means that BPH should be already at an advanced stage to be diagnosed;
for many veterinarians, who are reluctant to perform this examination in the presence of the owner.

Radiographic examination has several limitations, which nowadays, mean that it is an imaging examination of secondary importance:
Radiography overestimates the size of the prostate. Surrounding organs make the delineation of the prostate difficult: colon, abdominal wall and pericapsular adipose tissue (Feeney D A, Johnston G R, Klausner J S, et al.: Canine prostatic disease-comparison of ultrasonographic appearance with morphologic and microbiologic findings: 30 cases (1981-1985). J Am Vet Med Assoc 1987; 190: 1027-1034).
BPH is diagnosed when the prostate diameter is greater than 70% of the distance from the post-sacrum promontory to the pubis tip. However, this 70% standard, which is used in radiography, does not take the age and race of the dog into account. The prostate swells physiologically with the age of the dog. The shape of the pelvis varies between races.
The assessment of prostatic parenchyma is limited: only calcifications or cavities of larger sizes can be noticed.

Although ultrasonography is the examination of choice in the diagnosis (and screening) of BPH and in the follow up of a medically treated dog (with nearly systematic recurrence after several months), it sometimes seems to under-diagnose early BPH or over-diagnose a recurrence.

The study by Ruel (Ruel Y, Barthez P Y, Mailles A, Begon D: Ultrasonographic evaluation of the prostate in healthy intact dogs. Vet Radiol Ultrasound 1998; 39: 212-216) proposes a wide range of normal sizes for the prostate. Indeed, in this study, 14% of dogs considered to be normal have a prostatic parenchyma with a glandulo-cystic appearance (as a result of hyperplasia). Finally, the study does not take into account the influence of race on the size of the prostate.

The study by Atalan (Atalan G, Holt P E, Barr F J et al. Ultrasonographic estimation of prostatic size in canine cadavers. Res. In vet. Sci. 1999; 67: 7-15) emphasizes the limitations in reliability of such measurements. According to the ultrasonography technique (position of the probe, associated digital rectal examination), large deviations in the measurements are possible. The width (cross-section) is subject to greater variability: a 30° rotation of the probe axis, when viewed on its cross-section, results in a variation of up to 20% in the measurement of prostate width (Johnston S D, Kustritz M V R, Oison P N S. Benign Prostatic Hypertrophy/Hyperplasia. In: Canine and feline theriogeology. Philadelphia: WB Saunders, 2001, 340-341).

The difficulty in performing the measurements is mainly due to:
the inclination of the probe relative to the prostate axis: due to the presence of the penis (lateral inclination), the prostate partly engaged into the pelvic canal (cranio-caudal angle);
prostate contours which are sometimes difficult to distinguish: ultrasonography quality (probe frequency, etc.), prostate located beyond the probe's field of view;
incorrect position of the probe on the prostate: the major axis must be used. Displacement of the lateral probe (longitudinal section) can lead to measurement variations of up to 10%.

Thus, ultrasonography suffers from a lack of reliability (operator's subjectivity) when performing measurements. In addition, because of the lack of reproducibility of these measurements, monitoring of the size of the prostate becomes difficult over a period of several months: when should re-treatment be used?

A specific biological marker may solve these reliability and reproducibility problems while at the same time allowing an objective result to be obtained.

Such markers have been investigated in dog urine by comparing, in a proteomic study, samples originating from healthy and sick dogs (Gemeiner M & Teinfalt M, Determination of changes in the prostate of dogs (WO/2001/050132)), but this work has not been pursued.

The search for markers from seminal fluid has been the subject of many studies.

Unlike in most animal species, dog semen contains a very limited number of proteins (Dubé J Y, Frenette G, Chapdelaine P, Paquin R and Tremblay R R. Biochemical characteristics of the proteins secreted by dog prostate, a review. Exp Biol 1985; 43: 149-159).

Acid phosphatase, one of the conventional prostate markers in man, is 100 times less concentrated in the canine seminal fluid than in man (Dubé J Y, Frenette G, Chapdelaine P, Paquin R and Tremblay R R. Biochemical characteristics of the proteins secreted by dog prostate, a review. Exp Biol 1985; 43: 149-159); its enzymatic activity in dogs is not significantly different from that of dogs with BPH (Bell F W, Klausner J S, Hayden D W et al. Evaluation of serum and seminal plasma markers in the diagnosis of canine prostatic disorders. J Vet Intern Med. 1995; 9:149-153).

In 1984, Chapdelaine et al. (Chapdelaine P, Dubé J Y, Frenette G et al. Identification of arginine esterase as the major androgen-dependent protein secreted by dog prostate and preliminary molecular characterization in seminal plasma. J. Androl. 1984; 5: 206-210) identified the major component of seminal fluid in dogs, arginine esterase or CPSE (Canine Prostate Specific Antigen, SwissProt No P09582). CPSE belongs to the kallikrein family of proteases that are capable of synthesizing kinins from kininogen. These enzymes are involved in many physiological processes, including inflammation and blood pressure.

A radioimmunoassay was developed (Frenette G, Dubé J Y, Lacoste D & Tremblay R R Radioimmunoassay in blood plasma of arginine esterase: the major secretory product of dog prostate. The Prostate 1987; 10: 145-152) for assaying CPSE in healthy dogs.

CPSE assaying in healthy dogs and dogs with BPH is performed by immunoprecipitation followed by immunoblotting, (Bell F W, Klausner J S, Hayden D W et al. Evaluation of serum and seminal plasma markers in the diagnosis of canine prostatic disorders. J Vet Intern Med. 1995; 9: 149-153), but the methods used did not allow the authors to distinguish, in a statistically significant manner, dogs with prostatic disorders on the one hand, from healthy dogs, on the other. The results given in this study have very little specificity and little sensitivity. For example, for dogs with CPSE levels below 199 ng/ml, it is not possible to reliably determine whether these dogs are healthy or suffer from a prostatic disorder.

The treatments currently available for BPH include surgery or the introduction of hormone therapy. In the latter case, the molecules used are either (steroidal or non-steroidal) anti-androgens, or LHRH (or GnRH) agonists. Monitoring of the action of an LHRH agonist in healthy dogs by assaying serum CPSE has been described (Frenette G, Dubé J Y, Lacoste D & Tremblay. R R Radioimmunoassay in blood plasma of arginine esterase: the major secretory product of dog prostate. The Prostate 1987; 10: 145-152), but this monitoring is usually carried out by assessing the size of the prostate (by means of ultrasonography) and is therefore subject to the uncertainties associated with this technique.

### DISCLOSURE OF THE INVENTION

The invention relates to an analysis method for the in vitro diagnosis of BPH in dogs, using the level or concentration of Canine Prostate Specific Esterase (CPSE) contained in biological fluids as a BPH marker. This method also uses biological molecules that specifically bind CPSE, such as specific polyclonal and/or monoclonal anti-CPSE antibodies, soluble receptors or aptamers.

The higher the CPSE level in the biological fluids of dogs, the greater the development of BPH. However, there is a threshold or reference value below which the dog can be considered, in a sufficiently reliable manner for the method to be used by veterinarians, as non-BPH, and on the contrary, above which the dog is declared to be suffering from BPH.

By non-BPH, is meant a dog with no BPH, but suffering from another prostatic disorder, such as prostatitis and/or adenocarcinoma of the prostate, or even a healthy dog.

The invention enables reliable results to be obtained, especially in dogs with blood levels of CPSE below 189 ng/ml.

More particularly, the invention relates to an analysis method for the in vitro analysis of BPH in a dog, using the CPSE contained in biological fluid samples collected from the dog as well as biological molecules that specifically bind CPSE, characterized in that:

a. a biological fluid sample is collected from a dog;

b. a Canine Prostate Specific Esterase (CPSE) concentration in said sample is measured;

c. said CPSE concentration is compared with a reference value; this value being between about 34 ng/ml and about 102.4 ng/ml for serum or plasma, preferably about 61 ng/ml;

d. the CPSE concentration is used as a BPH marker; and

e. BPH is diagnosed in the dog for CPSE concentrations measured in said samples, which are above said reference value.

Thus, advantageously, this method can diagnose prostatic hyperplasia in dogs while distinguishing it from prostatitis and/or prostate adenocarcinoma and the normal prostate.

The invention also relates to a process using the analysis method according to the invention for the follow up of a therapeutic treatment of BPH, that is, the follow up of the involution of the prostate during a treatment with anti-androgens, and for adapting the treatment of the animal accordingly (thus enabling the following question to be answered: when should re-treatment be used?).

The invention also relates to the use of an in vitro diagnostic kit comprising biological molecules that specifically bind CPSE for assaying the CPSE contained in the dog's biological fluids, for diagnosing BPH in dogs with a serum or plasma CPSE level between about 34 ng/ml and about 102.4 ng/ml, preferably between about 61 ng/ml and about 102.4 ng/ml, or following up the therapeutic treatment of a dog suffering from BPH.

The invention also relates to an in vitro diagnostic kit for diagnosing BPH in dogs or for the follow up of the treatment of a dog suffering from BPH. More particularly, said diagnostic kit comprises biological molecules that specifically bind to CPSE for assaying the CPSE contained in the biological fluids of dogs in order to diagnose BPH in dogs with a serum CPSE level between about 34 ng/ml and about 102.4 ng/ml, preferably between about 61 ng/ml and about 102.4 ng/ml; or for the follow up of the therapeutic treatment of a dog suffering from BPH, characterized in that the biological molecules that specifically bind CPSE are conjugated to radioisotopes, fluorescent molecules, luminescent molecules, enzymes, latex particles, metal colloids, and magnetic particles.

According to a first embodiment of the invention, the concentration of CPSE contained in the biological fluids collected from the dog (urine, serum, plasma, etc.) is used as a BPH marker. The analyses and studies conducted by the inventors and described below have identified a threshold or a reference value for this concentration. Thus, a dog is considered to have BPH when the level of serum or plasma CPSE in the biological fluid exceeds 34 ng/ml, and is preferably close to 61 ng/ml.

Statistical analyses have shown that the CPSE threshold of 61 ng/ml is an optimal clinical threshold. This concentration corresponds to a test sensitivity of 97.1% and a specificity of 92.7%. Some variability around this clinical threshold value is allowed, so that the sensitivity or specificity are preferably greater than 80%, preferably greater than 90%, and even more preferably greater than 95%.

However, it was considered that an analysis method would be acceptable for practitioners if it had a sensitivity and specificity of approximately 80% or more. Thus, the CPSE concentration in the biological fluids of dogs can be used as a BPH marker, when dogs are considered, which have a sufficiently high CPSE level for statistical analysis of the data to allow a sensitivity or test specificity greater than 80% to be achieved.

As shown in Table 9 below, in the diagnosis method according to the invention, reference values in the range between 34 ng/ml, and 102.4 ng/ml can be employed when using both the serum and plasma samples.

With regard to blood samples, the plasma of which represents about 55% of the total volume, the reference values may be in the range between about 18 ng/ml and about 56 ng/ml when, in the diagnosis method according to the invention, blood samples are used.

Corresponding reference values in urine, seminal fluid, or any other biological fluid, that are proportional to the following values can be determined by those skilled in the art through routine experimentation within their reach.

According to the invention, the reference value is preferably 61 ng/ml for serum or plasma samples.

Advantageously, the analysis method according to the invention comprises an additional step in which BPH is distinguished from prostatitis and/or prostate adenocarcinoma. Preferably, said method allows BPH to be distinguished from prostatitis and prostate adenocarcinoma.

Further advantageously, when a dog is treated with an anti-androgen agent, the method according to the invention can determine the efficacy of the treatment based on CPSE concentration measurements performed at regular intervals.

According to a second embodiment, the invention relates to a process which uses an analysis method according to the invention. This process advantageously allows for the follow-up of the therapeutic treatment of BPH. This method comprises the steps of:
measuring the concentration of CPSE in a sample collected from biological fluids of the dog;
diagnose BPH by comparing the value of this concentration to the reference values;
treating the animal with an anti-androgen according to the procedure indicated in case of BPH by the manufacturer of this anti-androgen;
carrying out, at regular intervals after the treatment, measurements of the concentration of CPSE in the biological fluids of the animal.

The invention also relates to the use of an in vitro diagnostic kit comprising biological molecules, which specifically bind CPSE, for assaying the CPSE contained in the biological fluids of dogs for diagnosing BPH in dogs which have a serum CPSE level in the range between about 34 ng/ml and about 102.3 ng/ml, preferably in the range between about 61 ng/ml and 102.4 ng/ml, or for the following-up of the therapeutic treatment of a dog suffering from BPH. The biological molecules that specifically bind CPSE are advantageously polyclonal or monoclonal antibodies, soluble receptors or aptamers.

Said biological molecules that specifically bind CPSP are advantageously coupled to large molecules such as biotin, avidin, streptavidin.

Preferably, the biological molecules that specifically bind CPSE are immobilized onto a solid carrier, such as nitrocellulose or other polymeric membranes, latex carriers, or plastic materials.

The invention also relates to an in vitro diagnostic kit for either diagnosing BPH or for evolution or involution follow-up after or during treatment. The kit can reliably and advantageously be applied to cases where the dog has a CPSE blood level of less than 189 ng/ml.

This kit contains biological molecules that specifically bind CPSE for assaying the CPSE contained in the dog's biological fluids. Advantageously, these biological molecules may be specific polyclonal and/or monoclonal anti-CPSE antibodies, soluble receptors or aptamers. They are coupled to large molecules such as biotin, avidin, streptavidin, and are immobilized onto a solid carrier such as nitrocellulose or other polymer membranes, latex carriers, or plastic materials.

The biological molecules that specifically bind CPSE both in said kit and in the CPSE are conjugated to radioisotopes, fluorescent molecules, luminescent molecules, enzymes, latex particles, metal colloids, or magnetic particles.

### EXPERIMENTS CARRIED OUT

For the purposes of their study, the inventors have conducted their analysis using the seminal fluid and prostatic serum collected from a dog, without this being a limitation. It is understood that the method according to the invention can be applied to samples collected from other biological fluids such as urine, blood plasma, lachrymal fluid, and salivary secretions.

Purification of CPSE:

CPSE is purified from prostatic fluid as described in a document by Isaacs W B (Isaacs W B, Shaper J H. Isolation and characterisation of the major androgen-dependent glycoprotein of canine prostatic fluid. 1983 J. Biol. Chem. 258: 6610-6615).

Preparation of Anti-CPSE Antiserum:

The polyclonal serum of rabbit anti-CPSE is prepared by immunizing New Zealand White rabbits against purified CPSE. The animals received 5 injections of 300 μg antigen each, on days D0, D14, D28, D42 and D56. The rabbits were killed 77 days after the first injection. Anti-CPSE antibodies are purified from the serum on a protein A column.

Immunoreactivity of Antiserum Analyzed by Western Blot:

Seminal fluid or blood from a dog suffering from BPH are collected, with the serum being obtained by blood centrifugation. Four microliters of each of these biological fluids are separated by SDS-PAGE electrophoresis under reducing or non-reducing conditions.

Electrotransfer of this gel to a nitrocellulose membrane is then performed. The latter is then dried and thereafter saturated.

Immunoblotting is performed on this membrane: it is immersed in a dilution of anti-CPSE antibodies for one hour, then developed with an anti-species antibody coupled to the alkaline phosphatase. The final step of development is performed with BCIP/NBT (3-bromo-4-chloro-5-indolyl phosphate and nitro blue tetrazolium).

Preparation of Monoclonal Anti-CPSE Antibodies:

Five Balb/c mice were injected with the purified CPSE. The serum titer of anti-CPSE was monitored using an ELISA assay. Once the titer had reached a sufficient level, the splenocytes were recovered and combined with SP20 murine myeloma cells. After a series of cloning and selection operations, the clones producing a monoclonal anti-CPSE antibody were selected and amplified.

Determination of the CPSE Concentration by Sandwich ELISA:

The CPSE concentration in the serum or plasma samples was determined by sandwich ELISA. 96-well microplates (Stripwell Costar/Corning, N.Y., USA) were coated overnight at +4° C. with monoclonal or polyclonal anti-CPSE antibodies, at a concentration of 5 μg/ml in a carbonate buffer at pH 9.6. After 4 washes, the wells were incubated for 1 hour at 37° C. with a saline phosphate buffer at pH 7.2, while adding 5% bovine serum albumin. Finally, the plates were incubated for 30 minutes at room temperature with a saline phosphate buffer at pH 7.2 to which 10% sucrose was added. Once emptied, the microplates were dried overnight under vacuum, and then packaged in the presence of a desiccant until use.

The assay of samples is carried out by dispensing 100 μm of serum or plasma (heparinized plasma or EDTA plasma) into each of the wells. After 1 hour at 37° C., the microplate is washed. 100 μl of monoclonal or polyclonal anti-CPSE antibodies conjugated to peroxidase are dispensed. The wells are incubated for 1 hr at 37° C. and washed again. The antibodies remaining attached to the walls are developed by adding 3,3′,5,5′tetramethyl benzidine. After completion of the reaction, the absorbance is read at 450 nm.

The ELISA assay was calibrated using purified CPSE. The assay provides a linear response for CPSE concentrations ranging between 0.39 and 20 ng/ml.

Determination of the CPSE Level by Membrane Immunochromatography

Serum CPSE was measured using the method described in patent application WO03062824. A first monoclonal or polyclonal anti-CPSE antibody is immobilized on a nitrocellulose membrane. The serum to be quantified is mixed with a second monoclonal or polyclonal anti-CPSE antibody conjugated to a fluorophore, and this mixture is deposited onto the nitrocellulose membrane. During migration, the CPSE antibody-fluorophore complex is fixed by the anti-CPSE immobilized on the membrane. The amount of fixed fluorescent anti-CPSE is proportional to the amount of CPSE in the sample. By measuring the emitted fluorescence it is thus possible to quantify the CPSE.

Statistical Analysis of Data:

The studied sample includes 89 dogs whose characteristics are listed in Table 1 below:

| | |
|---|---|
| Size of the study sample | 89 dogs |
| Dogs WITHOUT any clinical and/or | 48 dogs |
| BPH ultrasonography sign | |
| Dogs with adenocarcinoma | 2 dogs |
| Dogs with prostatitis | 5 dogs |
| Dogs WITH clinical and/or BPH | 34 dogs |
| ultrasonography signs | |

and are listed in detail in Table 2 below:

| Name | Age | | Status |
|---|---|---|---|
| L9 | | | BPH |
| L7 | | | Adenocarcinoma |
| D1 | 10 | years | Prostatitis |
| D3 | | | Prostatitis |
| D8 | 18 | months | Normal |
| BM3 | 8 | months | Normal |
| BM12 | 9 | months | Normal |
| BM28 | 11 | months | Normal |
| BM29 | 11.5 | months | Normal |
| BM7 | 8 | months | Normal |
| G4 | 10 | months | Normal |
| BM14 | 8 | months | Normal |
| BM1 | 14 | months | Normal |
| BM5 | 20 | months | Normal |
| BM8 | 9 | months | Normal |
| BM11 | 9 | months | Normal |
| BM30 | 11.5 | months | Normal |
| BM25 | 11 | months | Normal |
| BM23 | 11 | months | Normal |
| BM17 | 13.5 | months | Normal |
| BM20 | 12.5 | months | Normal |
| D16 | 8 | years | Prostatitis |
| BM19 | 12 | months | Normal |
| BM16 | 13 | months | Normal |
| BM18 | 12 | months | Normal |
| D9 | <24 | months | Normal |
| G1 | 17.5 | months | Normal |
| G3 | 17 | months | Normal |
| BM21 | 12.5 | months | Normal |
| BM10 | 17 | months | Normal |
| V3 | 14 | months | Normal |
| G2 | 17.5 | months | Normal |
| BM9 | 18 | months | Normal |
| BM27 | 11 | months | Normal |
| BM2 | 8 | months | Normal |
| BM13 | 9 | months | Normal |
| V5 | 14 | months | Normal |
| BM26 | 11 | months | Normal |
| BM22 | 11 | months | Normal |
| M15 | 2 | years | Normal |
| BM24 | 11 | months | Normal |
| BM6 | 5 | months | Normal |
| L17 | | | Prostatitis |
| D5 | 2 | years | Normal |
| | 9 | months | |
| V2 | 14 | months | Normal |
| V6 | 14 | months | Normal |
| D10 | <24 | months | Normal |
| V7 | 19 | months | Normal |
| L24 | | | Normal |
| BM15 | 16 | months | Normal |
| V1 | 14 | months | Normal |
| V4 | 14 | months | Normal |
| M24 | | | BPH |
| M4 | 9 | years | BPH |
| L23 | | | BPH |
| L15 | | | BPH |
| D15 | <2 | years | Normal |
| D19 | 15.5 | years | BPH |
| D2 | 13 | years | Adenocarcinoma |
| M7 | | | BPH |
| D11 | 13.5 | years | BPH |
| L3 | | | BPH |
| L3 | | | BPH |
| M5 | 12 | years | BPH |
| M22 | | | BPH |
| L1 | | | BPH |
| L19 | | | BPH |
| L4 | | | Prostatitis |
| P4 | | | BPH |
| D12 | 9 | years | BPH |
| M3 | | | BPH |
| D4 | 4.8 | years | Normal |
| D6 | 10 | years | BPH |
| P6 | | | BPH |
| D21 | 12 | years | BPH |
| D18 | 10 | years | BPH |
| D17 | 8.5 | years | BPH |
| M25 | | | BPH |
| M20 | | | BPH |
| L18 | | | BPH |
| M26 | | | BPH |
| L5 | | | BPH |
| P7 | | | BPH |
| L22 | | | BPH |
| M6 | | | BPH |
| M33 | | | BPH |
| M1 | | | BPH |
| D2 | 7 | years | BPH |
| M2 | | | BPH |
| D7 | | | BPH |

For these dogs, serum samples were collected, aliquoted and frozen at −80° C. until use.

Those dogs having the following prostate symptoms are considered as potentially suffering from BPH:
blood loss with a drop of blood on the sheath, between urinations (of prostatic origin)
hematuria
constipation
lameness of the hind
hematospermia
incontinence

This observation of symptoms may possibly be confirmed by additional tests, ultrasonography, and, for some, a histological examination in addition to ultrasonography.

Dogs are considered to be normal when their prostatic ultrasonography is normal.

Anti-Androgen Treatment Follow Up:

Five dogs with signs of BPH (see inclusion criteria above) were treated with osaterone acetate (YPOZANE®, Virbac). This molecule is an anti-androgen, which acts on the prostatic parenchyma. The CPSE level was measured at days D15 and D0. The dogs were treated as recommended and were given from 0.25-0.5 mg/kg/day of osaterone acetate, for a period of 7 days. Serum was collected prior to any treatment and after 15 days. The CPSE level was measured in the different samples.

Statistical Analysis of the Data

Serum CPSE concentrations in healthy dogs, which have been diagnosed with BPH, were compared to concentrations in healthy dogs without BPH, using a Wilcoxon test. A non-parametric analysis of variance on ranks was performed to compare the concentration values of the Adenocarcinoma, Normal and Prostatitis groups with the group of dogs with BPH. Since this test involves multiple comparisons, the p value was adjusted according to Dunnett's adjustment. p values above 0.01 are considered to be non-significant.

In order to check the specificity of purified anti-CPSE rabbit antibodies, four microliters of serum or seminal fluid was deposited on a SDS-containing acrylamid gel, and under reducing or non-reducing conditions. After migration, the proteins are transferred onto a nitrocellulose membrane. Once saturated, this membrane is incubated in the presence of purified anti-CPSE rabbit antibodies. After washing, the antibodies that remain attached are detected with anti-rabbit goat antibodies coupled with the alkaline phosphatase. Alkaline phosphatase is developed using a chromogenic substrate, namely BCIP/NBT.

The attached figures illustrate the results obtained, and are given by way of illustrative examples, to assist in the understanding of the invention. They have no limiting character.

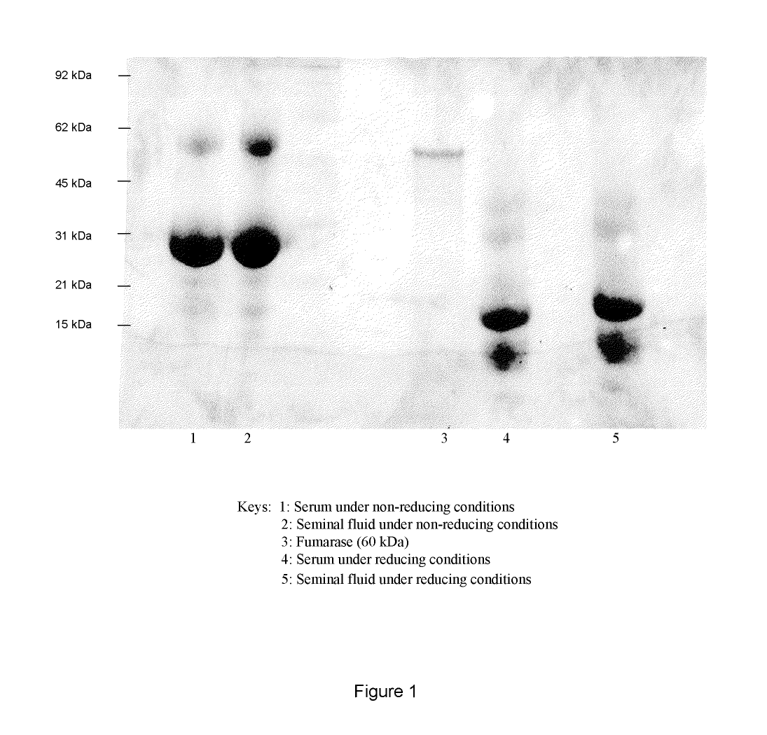

FIG. 1 shows the reactivity of immunoserum against prostatic fluid or serum from dogs with BPH (Keys: 1: Serum under non-reducing conditions; 2: Seminal fluid under non-reducing conditions; 3: Fumarase (60 kDa); 4: Serum under reducing conditions; 5: Seminal fluid under reducing conditions).

This figure shows, in tracks 1 and 2, a major band at 30 kDa corresponding to the non-reduced form of CPSE. A band at 60 kDa is also observed, and corresponds to an oligomer of CPSE (Isaac W B and Sharper H S Immunological localisation and quantification of the androgen-dependent secretory protease of the canine prostate. 1985 Endocrinology 117: 1512-1520). When the proteins are reduced, tracks 4 and 5, the two chains at 15 kDa (chain H) and 12-14 kDa (chain L) of CPSE are clearly observed.

It may thus be concluded that purified rabbit antibodies are indeed targeted against CPSE and do not recognize other serum proteins or proteins contained in the seminal fluid.

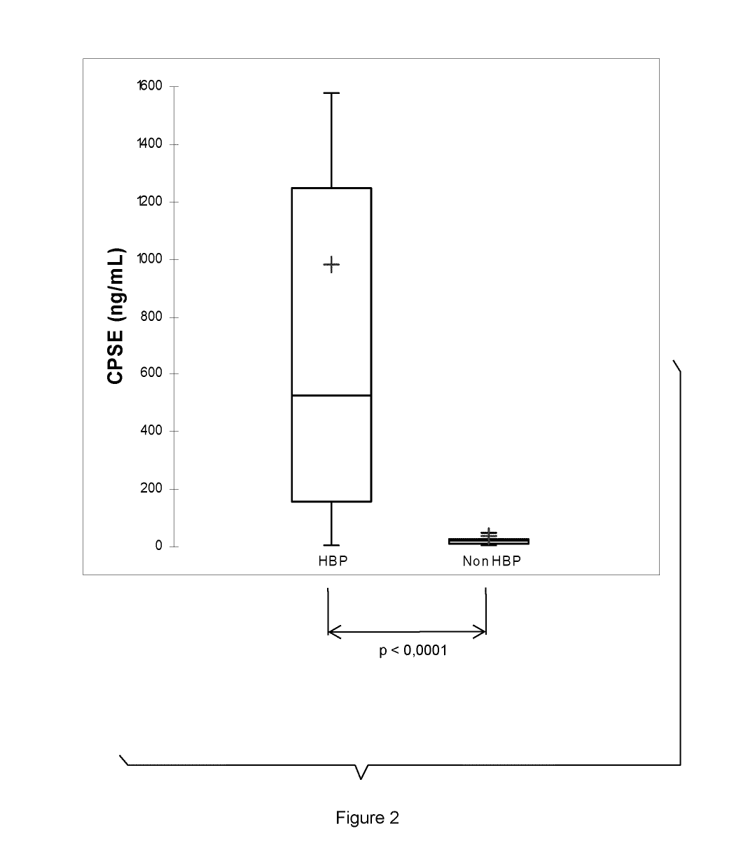

FIG. 2 shows the serum CPSE concentrations in dogs with clinical signs of BPH compared to those not showing these signs. These measurements were obtained using the ELISA assay described in the preceding sections.

The data is represented by a box-and-whisker plot. The (+) symbol denotes the mean, and (−) the median of the sample. The lower limit of the rectangle corresponds to the first quartile, and the upper limit, to the third quartile. The ends of the segments represent the first and ninth deciles.

Statistical analysis of data using a Wilcoxon test leads to p<0.0001, showing that the concentrations of CPSE in the group of dogs with clinical signs of BPH are significantly higher than in the group not showing these signs.

34 dogs are diagnosed with BPH, and 55 are diagnosed without BPH.

Analyzed variable: CPSE (ng/ml)

| | | | Standard | | | |
|---|---|---|---|---|---|---|
| | N | Mean | deviation | Median | Minimum | Maximum |
| BPH | 34 | 981.51 | 1282.01 | 527.05 | 5.70 | 6047.00 |
| Non-BPH | 55 | 38.79 | 82.31 | 22.00 | 5.90 | 507.70 |

This statistical analysis, as illustrated in Table 3 above, shows that the method of the invention is reliable and correctly distinguishes truly BPH dogs from non-BPH dogs.

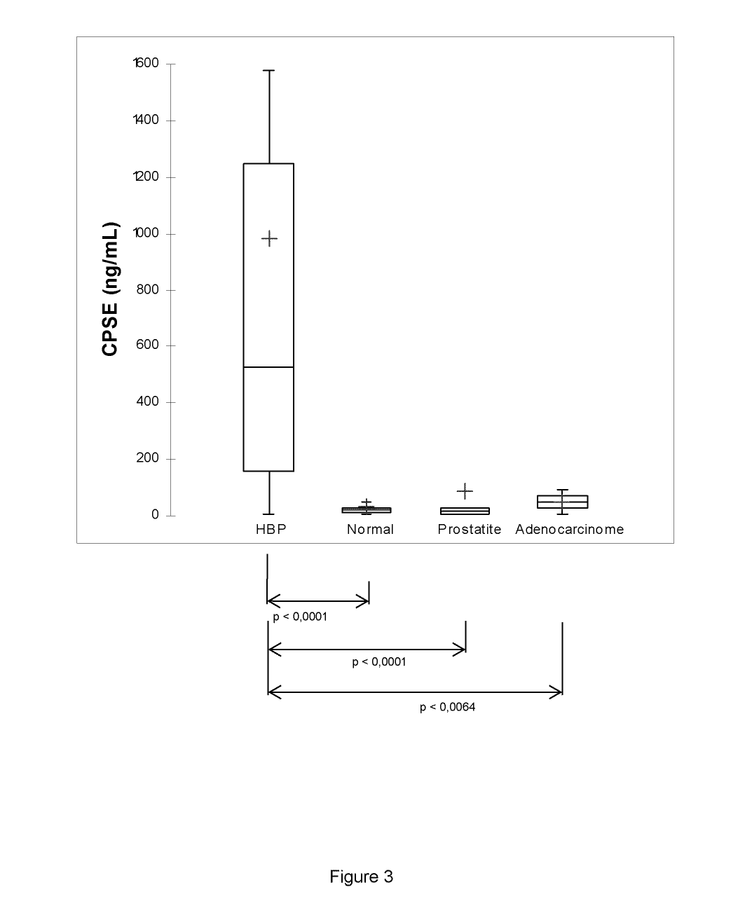

FIG. 3 shows the serum concentrations of CPSE in dogs with BPH compared to the CPSE level in dogs with other prostate diseases (prostatitis, adenocarcinoma), or in normal healthy dogs. These measurements were obtained according to the ELISA assay described in the materials and methods section.

The concentrations of CPSE in the different groups were compared using a non-parametric variance analysis on ranks. The values of p were adjusted according to Dunnett's adjustment.

The results (BPH vs. Adenocarcinoma p<0.0064; BPH vs. Prostatitis p<0.0001; BPH vs. Normal p<0.0001) indicate that the serum CPSE levels in dogs with adenocarcinoma, prostatitis, or in healthy dogs, are significantly lower than the CPSE levels in healthy dogs with BPH.

This statistical analysis shows that the method of the invention is reliable and correctly distinguishes dogs with BPH from those with prostatitis or carcinoma, or healthy dogs.

48 dogs have a normal prostate (n=48), 34 dogs show clinical signs of BPH (n=34), 5 dogs show signs of prostatitis (n=5) and 2 dogs were diagnosed with adenocarcinoma (n=2). See Table 4 below:

Analyzed variable: CPSE (ng/ml)

| | | | Standard | | Mini- | |
|---|---|---|---|---|---|---|
| | N | Mean | deviation | Median | mum | Maximum |
| Adenocarcinoma | 2 | 48.15 | 59.75 | 48.15 | 5.90 | 90.40 |
| NBPH | 34 | 981.51 | 1282.01 | 527.05 | 5.70 | 6047.00 |
| Normal | 48 | 33.25 | 71.32 | 22.40 | 6.50 | 507.70 |
| Prostatitis | 5 | 88.16 | 165.36 | 15.60 | 6.40 | 383.50 |

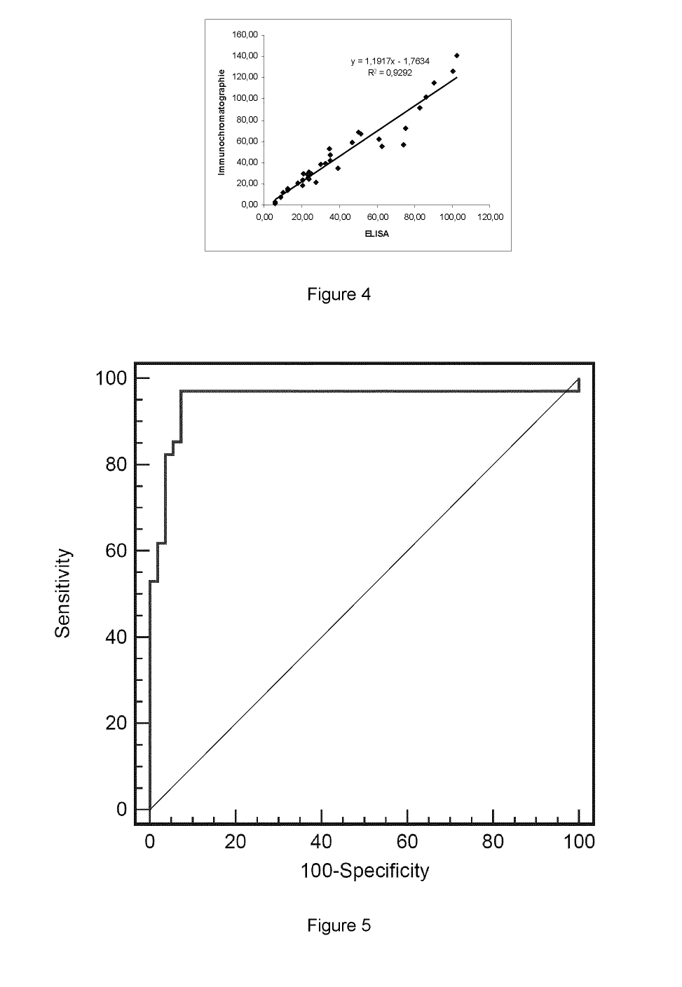

FIG. 4 shows that a correlation exists between the CPSE measurements obtained by ELISA and by immunochromatography on a membrane.

The level of serum CPSE was measured using either ELISA or by quantitative immunochromatography on a membrane, as described in the preceding sections. The concentrations measured in the two systems are represented in this Figure. The value of r²=0.92 shows that both methods are highly correlated.

FIG. 5 illustrates a ROC (Receiver Operating Characteristic) analysis of the dog sample.

In order to determine the level of serum CPSE for diagnosing BPH with the highest confidence possible, the inventors analyzed a sample of 89 dogs for which all clinical and ultrasound data were known.

The ROC (Receiver Operating Characteristic) analysis of the sample, with a clinical decision threshold of 61 ng/ml, can diagnose BPH with a sensitivity of 97.1% and a specificity of 92.7%. Under these conditions, the Younden index (sensitivity+specificity−1) is equal to 0.898, thus demonstrating the relevance of the threshold.

However, the sensitivity or the specificity of 80% represents acceptable levels of confidence for the diagnosis. In the Table below, the inventors have repeated the ROC analysis using different clinical decision thresholds. It appears that it is possible to diagnose BPH with a sensitivity of 79% by using a threshold of 102.4 ng/ml.

Conversely, a diagnosis of BPH can be achieved with a specificity of 80% using as the clinical decision threshold the serum CPSE level of 34.8 ng/ml. See Table 5 below:

| | | | Serum or plasma |
|---|---|---|---|
| | Sensitivity | Specificity | CPSE level |
| Decrease in | 97.1% | 92.7% | 61 ng/ml |
| sensitivity | 94% | 93% | 62.8 ng/ml |
| | 88% | 93% | 75.4 ng/ml |
| | 85% | 93% | 82.6 ng/ml |
| | 80% | 96% | 102.4 ng/ml |
| Decrease in | 97% | 89% | 50 ng/ml |
| specificity | 97% | 86% | 39.3 ng/ml |
| | 97% | 80% | 34.8 ng/ml |

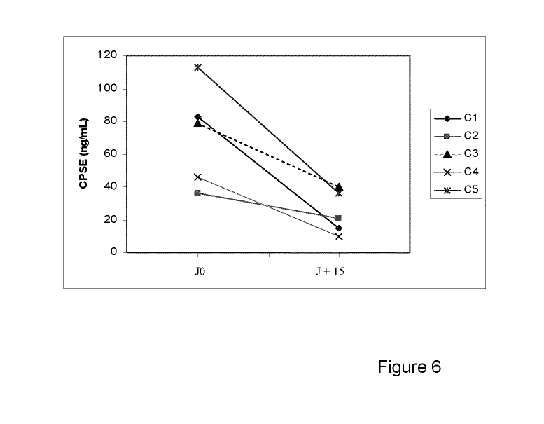

FIG. 6 shows the change in serum CPSE levels during the follow up of a treatment with an anti-androgen.

Dogs with signs of PBH were treated with osaterone acetate, as recommended by the manufacturer. The serum CPSE level in these dogs was measured before the treatment and after 15 days. See Tables 6-8 below.

TABLE 6

| | Date of 1st | Date of 2nd |
|---|---|---|
| Case No | sampling | sampling |
| 1 | Jan. 08, 2009 | Jan. 23, 2009 |
| 2 | Jan. 19, 2009 | Feb. 03, 2009 |
| 3 | Jan. 19, 2009 | Feb. 03, 2009 |
| 4 | Jan. 19, 2009 | Feb. 03, 2009 |
| 5 | Jan. 19, 2009 | Feb. 03, 2009 |

TABLE 7
CPSE (ng/ml)

| | Visit 1 | Visit 2 |
|---|---|---|
| C1 | 83 | 15 |
| C2 | 36 | 21 |
| C3 | 79 | 40 |
| C4 | 46 | 10 |
| C5 | 113 | 36 |

TABLE 8
CPSE (ng/ml)

| | D0 | D + 15 |
|---|---|---|
| Dog 1 | 83 | 15 |
| Dog 2 | 36 | 21 |
| Dog 3 | 79 | 40 |
| Dog 4 | 46 | 10 |
| Dog 5 | 113 | 36 |

These results show a decrease in the serum CPSE level between the time when the treatment with osaterone acetate had not yet begun, and the time when the treatment was completed. In parallel, a return to a normal size of the prostate was observed in all of these dogs, by means of digital rectal examination.

The serum level of CPSE can be used to follow up BPH treatment with an anti-androgen. This level is therefore an indicator of the efficacy of the treatment.

TABLE 9
Comparison of serum and plasma samples

CPSE (ng/ml)

| | | Heparinized |
|---|---|---|
| Sample | Serum | plasma |
| 1 | 36 | 37 |
| 2 | 44 | 48 |
| 3 | 33 | 33 |
| 4 | 48 | 53 |
| 5 | 31 | 30 |
| 6 | 60 | 60 |
| 7 | 16 | 16 |
| 8 | 35 | 38 |
| 9 | 42 | 46 |
| 10 | 29 | 29 |
| 11 | 18 | 19 |
| 12 | 122 | 118 |
| 13 | 153 | 172 |
| 14 | 146 | 148 |
| 15 | 129 | 129 |
| 16 | 121 | 136 |
| 17 | 30 | 31 |

The above Table shows that the CPSE concentrations are substantially identical for serum and heparinized plasma samples.

1. An analysis method for the in vitro diagnosis of benign prostatic hyperplasia (BPH) in a dog, using the CPSE contained in biological fluid samples collected from the dog as well as biological molecules that specifically bind CPSE, characterized in that:
a. a biological fluid sample is collected from a dog;
b. a Canine Prostate Specific Esterase (CPSE) concentration in said sample is measured;
c. said measured CPSE concentration is compared with a reference value, this value being between about 34 ng/ml and about 102.4 ng/ml for serum or plasma;
d. the CPSE concentration is used as a BPH marker; and
e. BPH is diagnosed in the dog for CPSE concentrations measured in said samples, which are above said reference value.

2. The analysis method according to claim 1, wherein the reference value is about 61 ng/ml for serum or plasma.

3. The analysis method according to claim 1, wherein at step e), BPH is distinguished from prostatitis and/or adenocarcinoma of the prostate.

4. The analysis method according to claim 1, wherein at step d), the reference value is a value between about 18 and about 56 ng/ml for blood.

5. The analysis method according to claim 1, wherein it allows prostate involution to be followed up during a treatment with anti-androgens, and said treatment to be adapted according to the animal.

6. The analysis method according to claim 1, wherein the biological molecules used that specifically bind CPSE are a polyclonal antibody.

7. The analysis method according to claim 1, wherein the biological molecules used that specifically bind CPSE are a monoclonal antibody.

8. The analysis method according to claim 1, wherein the biological molecules used that specifically bind CPSE are soluble receptors or aptamers.

9. The analysis method according to claim 1, wherein the CPSE concentration is measured either by using the ELISA method or by quantitative immunochromatography on a membrane.

10. Method of diagnosing BPH in a dog, comprising using an in vitro diagnostic kit comprising biological molecules that specifically bind CPSE for assaying the CPSE contained in the dog's biological fluids for diagnosing BPH in the dog with a serum or plasma CPSE level between about 34 ng/ml and about 102.4 ng/ml.

11. The method according to claim 10, wherein the serum or plasma CPSE level is between about 61 ng/ml and 102.4 ng/ml.

12. The method according to claim 10, wherein the biological molecules that specifically bind CPSE are polyclonal or monoclonal antibodies, soluble receptors or aptamers.

13. The method according to claim 10, wherein the biological molecules that specifically bind CPSE are coupled with large molecules of the biotin, avidin, streptavidin type.

14. The method according to claim 10, wherein the biological molecules that specifically bind CPSE are immobilized onto a solid carrier.

15. The method according to claim 10, wherein the biological molecules that specifically bind CPSE are immobilized onto nitrocellulose or other polymeric membranes, latex carriers, or plastic materials.

16. The method according to claim 10, for the follow up of the therapeutic treatment of a dog with BPH.

17. An in vitro diagnostic kit comprising biological molecules that specifically bind CPSE in order to assay the CPSE contained in the dog's biological fluids for diagnosing BPH in the dog with a serum or plasma CPSE level between about 34 ng/ml and about 102.4 ng/ml, characterized in that the biological molecules that specifically bind CPSE are conjugated to radioisotopes, fluorescent molecules, luminescent molecules, enzymes, latex particles, metal colloids or magnetic particles.

18. The in vitro diagnostic kit according to claim 17, wherein the serum or plasma CPSE level is between about 61 ng/ml and about 102.4 ng/ml.

19. The in vitro diagnostic kit according to claim 17, for the follow up of the therapeutic treatment of a dog with BPH.
